(19) United States
(12) Patent Application Publication
(10) Pub. No.: US 2007/0065490 A1
(43) Pub. Date: Mar. 22, 2007

(54) Substrates and compounds bonded thereto

(76) Inventors: Mark S. Schaberg, Lake Elmo, MN (US); Karen B. Geahigan, North Bend, WA (US); Cary A. Kipke, Woodbury, MN (US); Brinda B. Lakshmi, Woodbury, MN (US); James K. Young, Austin, TX (US)

Correspondence Address: 3M INNOVATIVE PROPERTIES COMPANY, PO BOX 33427, ST. PAUL, MN, 55133-3427, US

(21) Appl. No.: 10/596,925

(22) PCT Filed: Dec. 28, 2004
(86) PCT No.: PCT/US04/43852

§ 371 Date: Jun. 29, 2006

Related U.S. Application Data
(60) Provisional application No. 60/533,162, filed on Dec. 30, 2003.

(51) Int. Cl. G01N 33/53; G01N 33/554; C12M 1/34

(52) U.S. Cl. 424/443; 435/7.1; 435/287.2; 435/7.32

(57) ABSTRACT

Immobilization substrates and tethering compounds compatible with such substrates are described. In one aspect, the invention provides an article comprising: a substrate having a first surface and a second surface; a triazine tethering group affixed to the first surface of the substrate, the triazine tethering group comprising a reaction product of a functional group on the first surface of the substrate with a triazine tethering compound. A method of immobilizing a nucleophile-containing material to a substrate is also described.

### FIELD

This invention relates to articles comprising a substrate having a tethering group affixed thereto and to methods for immobilizing a nucleophile-containing material to the substrate through the tethering group.

### BACKGROUND

The covalent attachment of biologically active molecules to the surface of a substrate can be useful in a variety of applications such as in diagnostic devices, affinity separations, high throughput DNA sequencing applications, the clean-up of polymerase chain reactions (PCR), and the like. Immobilized biological amines, for example, can be used for the medical diagnosis of a disease or genetic defect or for detection of various biomolecules.

The modification of solid supports (e.g. particulate chromatography supports) by introduction of reactive functional groups for the immobilization of any of a variety of ligands is known. The attachment of a nucleophile (e.g., NH₂, SH, OH, etc.) to a substrate may be achieved through the use of tethering compounds. A tethering compound has at least two reactive functional groups separated by a linking group. One of the functional groups provides a means for anchoring the tethering compound to a substrate or support by reacting with a complementary functional group on the surface of the substrate. A second reactive functional group can be selected to react with the nucleophile-containing material. Supports containing hydroxyl groups (e.g. cellulose, cross-linked dextrans, wool, and polyvinyl alcohol) may be treated with cyanuric chloride (trichlorotriazine) for the attachment of enzymes, antigens, and antibodies. Hydroxyl-containing supports such as Sepharose may be reacted with trichlorotriazine (TCT) which may then bind one or more nucleophiles. Solid nylon beads derivatized with cyanuric chloride have been used for oligonucleotide based hybridization assays. TCT coated paper and nylon membranes have also demonstrated utility in transfer hybridization experiments of DNA, RNA, and proteins.

Known tethering compounds are typically highly reactive with nucleophile-containing materials including biological materials. But, the reaction of the tethering compounds to nucleophile-containing materials may compete with other reactions, such as the hydrolysis of the tethering compound, when reactions with nucleophiles are conducted in aqueous solutions. Hydrolysis can result in incomplete or inefficient immobilization of the nucleophile-containing materials on a substrate.

There is a need for improved immobilization substrates and for tethering compounds compatible with such substrates. Accordingly, it is desired to provide supports and tethering compounds that are useful for ligand immobilization in any of a variety of applications.

### SUMMARY

The invention provides articles useful as immobilization substrates and methods for immobilizing a nucleophile-containing material to a substrate. In one aspect, the invention provides an article comprising: a substrate having a first surface and a second surface; a triazine tethering group affixed to the first surface of the substrate, the triazine tethering group comprising a reaction product of a functional group on the first surface of the substrate with a triazine tethering compound.

In another aspect, the invention provides a method of immobilizing a nucleophile-containing material to a substrate, the method comprising:

Selecting a triazine tethering compound;

Providing a substrate having a complementary functional group capable of reacting with the triazine tethering compound;

Preparing a substrate-attached triazine tethering group by reacting the triazine tethering compound with the complementary functional group on the substrate resulting in an ionic bond, covalent bond, or combinations thereof; and

Reacting the substrate-attached triazine tethering group with a nucleophile-containing material to tether the nucleophile-containing material to the substrate.

Certain terms used in the description of the invention will be understood to have the meanings set forth below:

As used herein, the term “acyl” refers to a monovalent group of formula —(CO)R where R is an alkyl group and where (CO) used herein indicates that the carbon is attached to the oxygen with a double bond.

As used herein, the term “acyloxy” refers to a monovalent group of formula —O(CO)R where R is an alkyl group.

As used herein, the term “acyloxysilyl” refers to a monovalent group having an acyloxy group attached to a Si (i.e., Si—O(CO)R where R is an alkyl). For example, an acyloxysilyl can have a formula —Si[O(CO)R]₃₋ₙLₙ where n is an integer of 0 to 2 and L is a halogen or alkoxy. Specific examples include —Si[O(CO)CH₃]₃, —Si[O(CO)CH₃]₂Cl, or —Si[O(CO)CH₃]Cl₂.

As used herein, the term “alkoxy” refers to a monovalent group of formula —OR where R is an alkyl group.

As used herein, the term “alkoxycarbonyl” refers to a monovalent group of formula —(CO)OR where R is an alkyl group.

As used herein, the term “alkoxysilyl” refers to a group having an alkoxy group attached to a Si (i.e., Si—OR where R is an alkyl). For example, an alkoxysilyl can have a formula —Si(OR)₃₋ₙ(L^{a})ₙ where n is an integer of 0 to 2 and L^{a} is a halogen or acyloxy. Specific examples include —Si(OCH₃)₃, —Si(OCH₃)₂Cl, or —Si(OCH₃)Cl₂.

As used herein, the term “alkyl” refers to a monovalent radical of an alkane and includes groups that are linear, branched, cyclic, or combinations thereof. The alkyl group typically has 1 to 30 carbon atoms. In some embodiments, the alkyl group contains 1 to 20 carbon atoms, 1 to 10 carbon atoms, 1 to 6 carbon atoms, or 1 to 4 carbon atoms. Examples of alkyl groups include, but are not limited to, methyl, ethyl, n-propyl, isopropyl, n-butyl, isobutyl, tert-butyl, n-pentyl, n-hexyl, cyclohexyl, n-heptyl, n-octyl, and ethylhexyl.

As used herein, the term “alkyl disulfide” refers to a monovalent group of formula —SSR where R is an alkyl group.

As used herein, the term “alkylene” refers to a divalent radical of an alkane. The alkylene can be straight-chained, branched, cyclic, or combinations thereof. The alkylene typically has 1 to 200 carbon atoms. In some embodiments, the alkylene contains 1 to 100, 1 to 80, 1 to 50, 1 to 30, 1 to 20, 1 to 10, or 1 to 4 carbon atoms. The radical centers of the alkylene can be on the same carbon atom (i.e., an alkylidene) or on different carbon atoms.

As used herein, the term “aralkyl” refers to a monovalent radical of the compound R—Ar where Ar is an aromatic carbocyclic group and R is an alkyl group.

As used herein, the term “aralkylene” refers to a divalent radical of formula —R—Ar— where Ar is an arylene group and R is an alkylene group.

As used herein, the term “aryl” refers to a monovalent aromatic carbocyclic radical. The aryl can have one aromatic ring or can include up to 5 carbocyclic ring structures that are connected to or fused to the aromatic ring. The other ring structures can be aromatic, non-aromatic, or combinations thereof. Examples of aryl groups include, but are not limited to, phenyl, biphenyl, terphenyl, anthryl, naphthyl, acenaphthyl, anthraquinonyl, phenanthryl, anthracenyl, pyrenyl, perylenyl, and fluorenyl.

As used herein, the term “arylene” refers to a divalent radical of a carbocyclic aromatic compound having one to 5 rings that are connected, fused, or combinations thereof. In some embodiments, the arylene group has up to 5 rings, up to 4 rings, up to 3 rings, up to 2 rings, or one aromatic ring. For example, the arylene group can be phenylene.

As used herein, the term “azido” refers to a group of formula —N₃.

As used herein, the term “aziridinyl” refers to a cyclic monovalent radical of aziridine having the formula 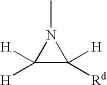
where R^{d} is hydrogen or alkyl.

As used herein, the term “benzotriazolyl” refers to a monovalent group having a benzene group fused to a triazolyl group. The formula for a benzotriazolyl group is C₆H₄N₃—.

As used herein, the term “carbonyl” refers to a divalent group of formula —(CO)—.

As used herein, the term “carbonylimino” refers to a divalent group of the formula —(CO)NR⁴— where R⁴ is hydrogen, alkyl, or aryl.

As used herein, the term “carbonyloxy” refers to a divalent group of formula —(CO)O—.

As used herein, the term “carbonyloxycarbonyl” refers to a divalent group of formula —CO)O(CO)—. Such a group is part of an anhydride compound.

As used herein, the term “carbonylthio” refers to a divalent group of formula —(CO)S—.

As used herein, the term “carboxy” refers to a monovalent group of formula CO)OH.

As used herein, the term “chloroalkyl” refers to an alkyl having at least one hydrogen atom replaced with a chlorine atom.

As used herein, the term “cyano” refers to a group of formula —CN.

As used herein, the term “disulfide” refers to a divalent group of formula —S—S—.

As used herein, the term “ethylenically unsaturated” refers to a monovalent group having a carbon-carbon double bond of formula —CY═CH₂ where Y is hydrogen, alkyl, or aryl.

As used herein, the term “fluoroalkyl” refers to an alkyl having at least one hydrogen atom replaced with a fluorine atom.

As used herein, the term “haloalkyl” refers to an alkyl having at least one hydrogen atom replaced with a halogen selected from F, Cl, Br, or I. Perfluoroalkyl groups are a subset of haloalkyl groups.

As used herein, the term “halocarbonyloxy” refers to a monovalent group of formula —O(CO)X where X is a halogen atom selected from F, Cl, Br, or I.

As used herein, the term “halocarbonyl” refers to a monovalent group of formula —(CO)X where X is a halogen atom selected from F, Cl, Br, or I.

As used herein, the term “halosilyl” refers to a group having a Si attached to a halogen (i.e., Si—X where X is a halogen). For example, the halosilyl group can be of formula —SiX₃₋ₙ(L^{b})ₙ where n is an integer of 0 to 2 and L^{b} is selected from an alkoxy, or acyloxy. Some specific examples include the groups —SiCl₃, —SiCl₂OCH₃, and —SiCl(OCH₃)₂.

As used herein, the term “heteroalkylene” refers to a divalent alkylene having one or more carbon atoms replaced with a sulfur, oxygen, or NR^{d} where R^{d} is hydrogen or alkyl. The heteroalkylene can be linear, branched, cyclic, or combinations thereof and can include up to 400 carbon atoms and up to 30 heteroatoms. In some embodiments, the heteroalkylene includes up to 300 carbon atoms, up to 200 carbon atoms, up to 100 carbon atoms, up to 50 carbon atoms, up to 30 carbon atoms, up to 20 carbon atoms, or up to 10 carbon atoms.

As used herein, the term “hydroxy” refers to a group of formula —OH.

As used herein, the term “isocyanato” refers to a group of formula —NCO.

As used herein, the term “mercapto” refers to a group of formula —SH.

As used herein, “nucleophile” or “nucleophile-containing material” refers to moieties with reactive oxygen, sulfur and/or nitrogen containing groups such as substituted amino groups. Examples of nucleophile-containing materials include those with moieties such as amino, alkyl or aryl substituted amino, alkylamino, arylamino, oxyalkyl, oxyaryl, thioalkyl, and thioaryl groups, residues of dyestuffs containing amino groups such as nitro-dyestuffs, azo-dystuffs, including thiazole dystuffs, acridine-, oxyazine-, thiazine- and azine dyestuffs, indigoids, aminoanthraquinones, aromatic diamines, aminophenols, aminonaphthols and N and O-acidyl or alkyl, aralkyl or aryl derivatives of these, nitramines, thiophenols, or amino mercaptans. Exemplary nucleophile-containing material include the following moieties: OCH2COOH; NHCH₂COOH; SCH₂COOH; NHC₂H₄SO₃H; OC₄H₈N(C₂H₅)₃; NHC₆H₄SO₃H; OC₆H₄COOH; SC₆H₄COOH; NHC₂H₄OH; OC₂H₄OH; and NHC₃H₆NH(C₂H₄OH)₂.

As used herein, the term “oxy” refers to a divalent group of formula —O—.

As used herein, the term “perfluoroalkyl” refers to an alkyl group in which all of the hydrogen atoms are replaced with fluorine atoms. Perfluoroalkyl groups are a subset of fluoroalkyl groups.

As used herein, the term “phosphato” refers to a monovalent group of formula —OPO₃H₂.

As used herein, the term “phosphono” refers to a monovalent group of formula —PO₃H₂.

As used herein, the term “phosphoramido” refers to a monovalent group of formula —NHPO₃H₂.

As used herein, the term “primary aromatic amino” refers to a monovalent group of formula —ArNH₂ where Ar is an aryl group.

As used herein, the term “secondary aromatic amino” refers to a monovalent group of formula —ArNR^{h}H where Ar is an aryl group and R^{h} is an alkyl or aryl.

As used herein, the term “tertiary amino” refers to a group of formula —NR₂ where R is an alkyl.

As used herein, the term “tethering compound” refers to a compound that has at least two reactive groups. One of the reactive groups can react with a complementary functional group on the surface of a substrate to secure the compound to the substrate and thus form a tethering group. Another reactive group on the compound can react either with a nucleophile-containing material, or another tethering compound (or a derivative or oligomer thereof) or another moiety capable of bonding with a nucleophile-containing material. The reaction of two reactive groups on the tethering compound results in the formation of a tethering group between the substrate and a nucleophile-containing material (e.g., an amine-containing material) that is immobilized on the substrate.

As used herein, the term “tethering group” refers to a group attached to a substrate that results from the reaction of a tethering compound with a complementary functional group on the surface of the substrate.

As used herein, the “triazine group” or “triazine moiety” refers to structures of the formula:

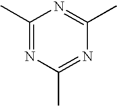

As used herein, “triazine tethering group” or “triazine tethering compound” refer to tethering groups or tethering compounds which include at least one triazine group.

The foregoing summary is not intended to be inclusive of all possible embodiments of the invention. Those skilled in the art will more fully appreciate the features and advantages of the invention upon consideration of the remainder of the disclosure including the Detailed Description of the Preferred Embodiment, the various Examples and the appended claims.

### DETAILED DESCRIPTION

The present invention provides constructions and methods for immobilizing nucleophile-containing materials to a substrate utilizing triazine tethering groups, as described herein. Compounds having reactive functional groups are described for use as tethering compounds between a substrate and at least one nucleophile-containing material.

A first reactive functional group on a triazine tethering compound provides a means of attaching the triazine tethering compound to a surface of a substrate. A second reactive functional group can be reacted with a nucleophile-containing material such as an amine functional protein, enzymes, other biomolecules or the like. Additional functional groups can be reacted with nucleophile-containing groups or can provide additional links to other moieties such as other triazine groups or other reactive moieties which may be simple or complex in their structures (e.g., branched, straight chain, etc.) and typically including additional reactive groups capable of bonding with nucleophile-containing groups.

In embodiments of the invention, triazine tethering compounds for bonding biological molecules to the surface of a substrate, may be of the general composition of Formula I: 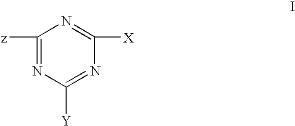
Wherein
X, Y and Z may be the same or different and may comprise (1) inorganic radicals or (2) organic or inorganic groups capable of forming a bond with a nucleophile-containing material, such as other triazine compounds including additional compounds of the Formula I. In some embodiments, X, Y and Z are the same.

In some embodiments, the triazine tethering compounds useful in the present invention include trichlorotriazine (TCT) wherein the X, Y and Z ligands of Formula I are all chlorine. In tethering the TCT to a substrate, at least one of the chlorines (e.g., the X ligand) is reacted with a moiety on the surface of a substrate to bond the triazine moiety to the substrate. When one of the chloride ligands reacts with the substrate, the remainder of the tethering compound comprises a substituted dichlorotriazine (DCT) in which the bond linking the triazine moiety to the substrate serves to anchor the triazine moiety to the substrate to form a triazine tethering group. The remaining unreacted chlorides on the TCT moiety remain capable of reacting with nucleophile-containing materials such as biologically active materials, derivatives of TCT, other organic or inorganic moieties, and the like.

In some embodiments of the invention, the triazine tethering groups may be derived solely from TCT molecules. In some embodiments, the triazine tethering groups are derived from compounds that may be considered to be oligomers of TCT, derivatives of TCT, oligomers of derivatized TCT, and the like. Referring to Formula I, triazine tethering groups derived solely from TCT are those compounds of Formula I wherein each of X, Y, and Z are chlorine.

Derivatives of TCT suitable for inclusion in the triazine tethering groups of the present invention include compounds of Formula I wherein at least one of X, Y or Z is a moiety that may be selected from any of a variety of monofunctional groups, difunctional groups or other multifunctional groups wherein the functional groups are typically nucleophiles. Such functional groups may be organic moieties that may be, in whole or in part, aliphatic (straight chain or branched chain) or aromatic. In some embodiments, the monofunctional, difunctional and/or multifunctional groups may be bonded to a triazine moiety prior to the attachment of the triazine moiety to the substrate. In some embodiments, the monofunctional, difunctional and/or multifunctional groups may be bonded to a triazine moiety after the triazine moiety has already been attached (e.g., bonded) to a substrate.

In embodiments where the triazine moiety is derived from TCT, reaction of the chlorines (X, Y and Z of Formula I are chlorine) is typically sequential and the reactivity of each chlorine depends on the number of chlorines remaining on the TCT molecule, the nature of the moiety (e.g., its nucleophilicity, steric factors) being reacted with the TCT and the reaction conditions (temperature, presence of water, the stoichiometry of the reactants, etc.). Where group X, for example, of Formula I is reacted with a moiety on the surface of a substrate to bond the triazine moiety to the substrate, the remaining unreacted groups Y and Z remain generally capable of reacting with nucleophile-containing materials such as monofunctional, difunctional and/or multifunctional moieties.

Monofunctional groups include a reactive group (e.g., nucleophiles) capable of reacting with one of the X, Y, or Z groups of the compounds of Formula I but generally do not include additional reactive groups. In some embodiments, monofunctional groups may comprise groups having one or more desired properties that are needed or desired in the substrates or the tethering groups of the present invention. Exemplary of suitable monofunctional groups include groups that render the reaction product hydrophilic or hydrophobic, groups that enhance solubility in certain solvents, groups that enhance molecular interactions, and the like. Examples include monofunctional organic alcohols, amines and mercaptans.

Difunctional groups may be linking groups in that they include a first reactive group that can react with a triazine moiety and a second reactive group that can react with another compound or moiety including another compound of Formula I such as TCT, for example. In some embodiments difunctional groups comprise linking groups that can link triazine moieties to one another to form a tethering group comprised of at least two triazine moieties connected to one another through the difunctional linking group. In such a configuration, the triazine moieties will include unreacted groups (e.g., unreacted X, Y or Z groups according to Formula I) capable of bonding with other nucleophile-containing materials such biologically active molecules, for example. In some embodiments, the unreacted groups may comprise chlorine on one, two or more triazine moieties tethered or linked together through one or more difunctional linking groups. At least some suitable difunctional moieties include compounds having two reactive groups such as two nucleophilic groups. Some specific difunctional groups include, for example, 4,7,10-trioxa-1,13-tridecane diamine, 1,6-hexanediamine, methyl-oxirane, p-phenylenediamine, 2-aminoethanol, 4,4-thiobisbenzenethiol, dimethyl-1,6-hexanediamine. Other difunctional moieties will be known to those of skill in the art, and the invention is not to be limited in any respect to the foregoing specific moieties.

Multifunctional moieties may also comprise linking groups in that they include a first reactive group that can react with a first triazine moiety bonded to a substrate, and second, third and possibly other additional reactive groups that can react with other compounds or moieties including other triazine moieties or compounds of Formula I (e.g., TCT). In some embodiments multifunctional groups include linking groups that can link two or more triazine moieties to one another to form a branched tethering group comprised of two or more triazine moieties linked together through the trifunctional linking group. In such a configuration, the triazine moieties will include unreacted groups (e.g., unreacted X, Y or Z groups according to Formula I) capable of bonding with other nucleophile-containing materials such as one or more biologically active molecules, for example. In some embodiments, the unreacted groups may comprise chlorines on one, two or more triazine moieties tethered or linked together through one or more multifunctional linking groups. Suitable multifunctional moieties include compounds having more than two reactive groups (e.g., nucleophilic groups). In some embodiments, the multifunctional moieties may be oligomeric or polymeric moieties. Some specific multifunctional moieties include, for example, hydrolyzed poly 2-ethyl-2-oxazoline (“Peox”), hydrolyzed 2-ethyl-4,5-dihydro-oxazole homopolymer, polyethylenimine (including linear and branched configurations), hydroxy substituted esters of polymethacrylates, hydroxy substituted esters of polyacrylates, polyvinyl alcohol, as well as other moieties known to those of ordinary skill.

It will be understood that the foregoing description should not be interpreted as limited to the specific monofunctional, difunctional or other multifunctional groups described herein. The present invention is intended to encompass tethering compounds and tethering groups that include at least one triazine moiety.

The invention provides articles that include a triazine tethering group, as described herein, attached to a substrate. The triazine tethering group is the reaction product of a triazine tethering compound and a complementary functional group on a surface of a substrate. The triazine tethering group may be represented by Formula I wherein the attachment of the triazine tethering group involves a reaction between the complementary functional group on the surface of the substrate with at least one of the groups X, Y and Z in compounds of Formula I. Once attached to a substrate, the triazine tethering group has at least one, typically two or more reactive groups that can react with a nucleophile-containing material to capture the material and tether it to the substrate.

The substrate is a solid phase material to which the triazine tethering compounds can be attached. The substrate is not soluble in a solution or solvent that might be used when attaching a triazine tethering compound to the surface of the substrate. Typically, a tethering compound is attached only to an outer portion (e.g., on or near the surface or within pores in the surface of the substrate) of the substrate while the remaining portions of the substrate are not modified during the process of attaching the tethering group to the substrate. If the substrate has groups “G” distributed throughout the substrate, only those groups in the outer portion are usually capable of reacting with a triazine moiety (e.g., by reacting with a group X, Y or Z of the compounds according to Formula I).

The substrate can have any useful form including, but not limited to, thin films, sheets, membranes, filters, nonwoven or woven fibers, hollow or solid beads or particles, fused or sintered beads or particles, bottles, plates, tubes, rods, pipes, or wafers. The substrates can be porous or non-porous, rigid or flexible, transparent or opaque, clear or colored, and reflective or non-reflective. Suitable substrate materials include, for example, polymeric materials, glasses, ceramics, metals, metal oxides, hydrated metal oxides, or combinations thereof.

The substrate can be a single layer of material or can have multiple layers of one or more materials. For example, the substrate can have one or more second layers that provide support for a first layer wherein the first layer of the substrate includes a complementary functional group capable of reacting with the triazine moiety (e.g., X, Y or Z groups of Formula I). The first layer is the outer layer of the substrate. In some embodiments, a surface of a first layer may be chemically modified or coated with another material to provide a complementary functional group capable of reacting with the triazine moiety.

Suitable polymeric substrate materials include, but are not limited to, polyolefins, polystyrenes, polyacrylates, polymethacrylates, polyacrylonitriles, poly(vinylacetates), polyvinyl alcohols, polyvinyl chlorides, polyoxymethylenes, polycarbonates, polyamides, polyimides, polyurethanes, phenolics, polyamines, amino-epoxy resins, polyesters, silicones, cellulose based polymers, polysaccharides, or combinations thereof. In some embodiments, the polymeric material is a copolymer prepared using a comonomer having a complementary functional group capable of reacting with the triazine moiety by reacting with a group X, Y or Z in compounds according to Formula I. For example, the comonomer can contain a carboxy, mercapto, hydroxy, amino, or alkoxysilyl group.

In some embodiments, suitable polymeric membrane materials include those resulting from thermally induced phase separation (“TIPS”) which is a phase inversion method in which an initially homogeneous polymer solution is cast and exposed to a cooler interface (e.g., a water bath or chilled casting wheel), and phase separation is induced in the solution film by lowering the temperature. Suitable TIPS films or membranes may possess a broad range of physical film properties and microscopic pore sizes. They may be relatively rigid or non-rigid substrates prepared from any of a variety of polymers. TIPS membranes made according to the teachings of U.S. Pat. Nos. 4,539,256, 5,120,594, and 5,238,623 are suitable for use in the invention. The TIPS membranes may comprise high density polyethylene (HDPE), polypropylene, polyvinylidenefluoride (PVDF), polyethylene-vinyl alcohol copolymer (e.g., available under the trade designation EVAL F101A from EVAL Company of America (EVALCA), Houston, Tex.), for example. The TIPS membrane may comprise a combination of materials such as the above mentioned HDPE or polypropylene membranes coated with a hydrophilic polymer (e.g., polyethylene-vinyl alcohol copolymer or EVAL), or the TIPS membrane may comprise a polypropylene support coated with a hydrophilic, strongly basic positively-charged coating such as polydiallyldimethylammonium chloride or a polymer incorporating quaternized dimethylaminoethylacrylate. The TIPS technology can provide a broad range of physical film properties having pore sizes in the micro- and ultra-filtration range. Combinations of materials may be used as a solid support member and the foregoing description is to be understood to include the aforementioned materials alone and in combination with other materials.

TIPS membranes generally provide a microporous structure with pores extending through the membrane having comprising a pore diameter within the range from about 80 nm to about 0.5 micron. One example of a suitable commercially available TIPS membrane for use in the invention is a HDPE membrane commercially available from 3M Company of St. Paul, Minn. and having features that include a pore size of about 0.09 um and a thickness of about 0.9 mil (0.023 mm). In some embodiments, a diamond like glass (DLG) coating may be applied to the TIPS substrate. The DLG coating may be applied using conventional or known techniques such as by a plasma deposition process like that described in EP 1 266 045 B1 (David et al). In the coating process, a DLG coating is typically applied over the entire surface of the TIPS membrane so that the DLG extends into the pores of the TIPS material. As mentioned, other materials may be used in the manufacture of a TIPS membrane, and a DLG coating may similarly be applied to such other materials in order to provide a suitable substrate for use in the present invention.

Suitable glass and ceramic materials for use as the substrate in articles of the invention include, for example, sodium, silicon, aluminum, lead, boron, phosphorous, zirconium, magnesium, calcium, arsenic, gallium, titanium, copper, or combinations thereof. Glasses typically include various types of silicate containing materials. In some embodiments, the substrate includes a layer of diamond-like glass such as that described in International Patent Application WO 01/66820 A1, the disclosure of which is incorporated herein in its entirety by reference thereto. Diamond-like glass is an amorphous material that typically includes carbon, silicon, and one or more elements selected from hydrogen, oxygen, fluorine, sulfur, titanium, or copper. Some diamond-like glass materials are formed from a tetramethylsilane precursor using a plasma process. A hydrophobic material can be produced that is further treated in an oxygen plasma to control the silanol concentration on the surface.

Diamond-like glass can be in the form of a thin film or in the form of a coating on another layer or material in the substrate. In some applications, the diamond-like glass can be in the form of a thin film having at least 30 weight percent carbon, at least 25 weight percent silicon, and up to 45 weight percent oxygen. Such films can be flexible and transparent. In some embodiments, the diamond-like glass is the outer layer of a multilayer substrate. In a specific example, the second layer (e.g., support layer) of the substrate is a polymeric material (e.g., a TIPS membrane) and the first layer is a thin film of diamond-like glass. The tethering group is attached to the surface of the diamond-like glass.

In some embodiments, the diamond-like glass is deposited on a layer of diamond-like carbon. For example, the second layer (e.g., support layer) may be a polymeric film or membrane having a layer of diamond-like carbon deposited on the polymer surface. A layer of diamond-like glass is deposited over the diamond-like carbon layer. The diamond-like carbon can, in some embodiments, function as a tie layer or primer layer between a polymeric layer and a layer of diamond-like glass in a multilayer substrate. For example, the multilayer substrate can include a polyimide or polyester layer, a layer of diamond-like carbon deposited on the polyimide or polyester, and a layer of diamond-like glass deposited on the diamond-like carbon. In another example, the multilayer substrate includes a stack of the layers arranged in the following order: diamond-like glass, diamond-like carbon, polyimide or polyester, diamond-like carbon, and diamond-like glass.

Diamond-like carbon films can be prepared, for example, from acetylene in a plasma reactor. Other methods of preparing such films are described U.S. Pat. Nos. 5,888,594 and 5,948,166 as well as in the article M. David et al., AlChE Journal, 37 (3), 367-376 (March 1991), the disclosures of which are incorporated herein by reference.

Metals, metal oxides, or hydrated metal oxides may also be suitable for use in substrates. Suitable materials for use in the present invention include, for example, gold, silver, platinum, palladium, aluminum, copper, chromium, iron, cobalt, nickel, zinc, and the like. The metal-containing material can be alloys such as stainless steel, indium tin oxide, and the like. In some embodiments, a metal-containing material is used in providing an upper or topmost layer of a multilayer substrate. For example, the substrate can have a polymeric second layer and a metal containing first layer. In one example, the second layer is a polymeric film and the first or uppermost layer is a thin film of gold. In other examples, a multilayer substrate includes a polymeric film coated with a titanium-containing layer which, in turn, is coated with a gold-containing layer. That is, the titanium layer can function as a tie layer or a primer layer for adhering the layer of gold to the polymeric film.

In other embodiments of a multi layer substrate for use in the invention, a silicon support layer is covered with a layer of chromium and then with a layer of gold. The chromium layer can improve the adhesion of the gold layer to the silicon layer.

The outer surface of the substrate will typically include a moiety or reactive group capable of reacting with a tethering compound that includes reactive groups comprising halogen, carboxy, halocarbonyl, halocarbonyloxy, cyano, hydroxy, mercapto, isocyanato, halosilyl, alkoxysilyl, acyloxysilyl, azido, aziridinyl, haloalkyl, tertiary amino, primary aromatic amino, secondary aromatic amino, disulfide, alkyl disulfide, benzotriazolyl, phosphono, phosphoroamido, phosphato, an ethylenically unsaturated group, or the like. In other words, the substrate is capable of reacting with one or more of X, Y or Z in compounds of Formula I (i.e., the substrate includes a complementary functional group to the group X, Y or Z). Substrates can include a support material that has been treated to provide an outer layer that includes a complementary functional group. The substrate can be prepared from any solid phase material known to have groups capable of reacting with the triazine moiety (e.g., X, Y or Z of Formula I) and is not limited to the following examples of suitable materials.

A carboxy group or a halocarbonyl group can react with a substrate having a hydroxy group to form a carbonyloxy-containing attachment group. Examples of substrate materials having hydroxy groups include, but are not limited to, polyvinyl alcohol, corona-treated polyethylene, hydroxy substituted esters of polymethacrylates, hydroxy substituted esters of polyacrylates, and a polyvinyl alcohol coating on a support material such as glass or polymer film.

A carboxy group or a halocarbonyl group can also react with a substrate having a mercapto group to form a carbonylthio-containing attachment group. Examples of substrate materials having a mercapto group include, but are not limited to, mercapto substituted esters of polyacrylates, mercapto substituted esters of polymethacrylates, and glass treated with a mercaptoalkylsilane.

Additionally, a carboxy group or a halocarbonyl group can react with a primary aromatic amino group, a secondary aromatic amino group, or a secondary aliphatic amino group to form a carbonylimino-containing attachment group. Examples of substrate materials having aromatic primary or secondary amino groups include, but are not limited to, polyamines, amine substituted esters of polymethacrylate, amine substituted esters of polyacrylate, polyethylenimines, and glass treated with an aminoalkylsilane.

A halocarbonyloxy group can react with a substrate having a hydroxy group to form an oxycarbonyloxy-containing attachment group. Examples of substrate materials having hydroxy groups include, but are not limited to, polyvinyl alcohol, corona-treated polyethylene, hydroxy substituted esters of polymethacrylates, hydroxy substituted esters of polyacrylates, and a polyvinyl alcohol coating on a support material such as glass or polymer film.

A halocarbonyloxy group can also react with a substrate having a mercapto group to form an oxycarbonylthio-containing attachment group. Examples of substrate materials having a mercapto group include, but are not limited to, mercapto substituted esters of polymethacrylates, mercapto substituted esters of polyacrylates, and glass treated with a mercaptoalkylsilane.

Additionally, a halocarbonyloxy group can react with a substrate having a primary aromatic amino group, a secondary aromatic amino group, or a secondary aliphatic amino group to form an oxycarbonylimino-containing attachment group. Examples of substrate materials having aromatic primary or secondary amino groups include, but are not limited to, polyamines, amine substituted esters of polymethacrylate, amine substituted esters of polyacrylate, polyethylenimines, and glass treated with an aminoalkylsilane.

A cyano group can react with a substrate having an azido group to form a tetrazinediyl-containing attachment group. Examples of substrates having azido groups include, but are not limited to, a coating of poly(4-azidomethylstyrene) on a glass or polymeric support. Suitable polymeric support materials include polyesters, polyimides, and the like.

A hydroxy group can react with a substrate having isocyanate group to form an oxycarbonylimino-containing attachment group. Suitable substrates having isocyanate groups include, but are not limited to, a coating of 2-isocyanatoethylmethacrylate polymer on a support material. Suitable support materials include glass and polymeric materials such as polyesters, polyimides, and the like.

A hydroxy group can also react with a substrate having a carboxy, carbonyloxycarbonyl, or halocarbonyl to form a carbonyloxy-containing attachment group. Suitable substrates include, but are not limited to, a coating of acrylic acid polymer or copolymer on a support material or a coating of a methacrylic acid polymer or copolymer on a support material. Suitable support materials include glass and polymeric materials such as polyesters, polyimides, and the like. Other suitable substrates include copolymers of polyethylene with polyacrylic acid, polymethacrylic acid, or combinations thereof.

A mercapto group can react with a substrate having isocyanate groups. The reaction between a mercapto group and an isocyanate group forms a thiocarbonylimino-containing attachment group. Suitable substrates having isocyanate groups include, but are not limited to, a coating of 2-isocyanatoethylmethacrylate copolymer on a support material. Suitable support materials include glass and polymeric materials such as polyesters, polyimides, and the like.

A mercapto group can also react with a substrate having a halocarbonyl group to form a carbonylthio-containing attachment group. Substrates having halocarbonyl groups include, for example, chlorocarbonyl substituted polyethylene.

A mercapto group can also react with a substrate having a halocarbonyloxy group to form an oxycarbonlythio-containing attachment group. Substrates having halocarbonyl groups include chloroformyl esters of polyvinyl alcohol.

Additionally, a mercapto group can react with a substrate having an ethylenically unsaturated group to form a thioether-containing attachment group. Suitable substrates having an ethylenically unsaturated group include, but are not limited to, polymers and copolymers derived from butadiene.

An isocyanate group can react with a substrate having a hydroxy group to form a oxycarbonylimino-containing attachment group. Examples of substrate materials having hydroxy groups include, but are not limited to, polyvinyl alcohol, corona-treated polyethylene, hydroxy substituted esters of polymethacrylates or polyacrylates, and a polyvinyl alcohol coating on glass or polymer film.

An isocyanate group can also react with a mercapto group to form a thiocarbonylimino-containing attachment group. Examples of substrate materials having a mercapto group include, but are not limited to, mercapto substituted esters of polymethacrylates or polyacrylates and glass treated with a mercaptoalkylsilane.

Additionally, an isocyanate group can react with a primary aromatic amino group, a secondary aromatic amino group, or a secondary aliphatic amino group to form a urea-containing attachment group. Suitable substrates having a primary or secondary aromatic amino group include, but are not limited to, polyamines, polyethylenimines, and coatings of an aminoalkylsilane on a support material such as glass or on a polymeric material such as a polyester or polyimide.

An isocyanate group can also react with a carboxy to form an O-acyl carbamoyl-containing attachment group. Suitable substrates having a carboxylic acid group include, but are not limited to, a coating of an acrylic acid polymer or copolymer or a coating of a methacrylic acid polymer or copolymer on a glass or polymeric support. Copolymers include, but are not limited to, copolymers that contain polyethylene and polyacrylic acid or polymethacrylic acid. Suitable polymeric support materials include polyesters, polyimides, and the like.

A halosilyl group, an alkoxysilyl group, or an acyloxysilyl group can react with a substrate having a silanol group to form a disiloxane-containing attachment group. Suitable substrates include those prepared from various glasses, ceramic materials, or polymeric material. These groups can also react with various materials having metal hydroxide groups on the surface to form a silane-containing linkage. Suitable metals include, but are not limited to, silver, aluminum, copper, chromium, iron, cobalt, nickel, zinc, and the like. In some embodiments, the metal is stainless steel or another alloy. Polymeric material can be prepared to have silanol groups. For example, commercially available monomers with silanol groups include 3-(trimethoxysilyl)propyl methacrylate and 3-aminoproplytrimethoxysilane available from Aldrich Chemical Co., Milwaukee, Wis.

An azido group can react, for example, with a substrate having carbon-carbon triple bond to form triazolediyl-containing attachment groups. An azido group can also react with a substrate having nitrile groups to form a tetrazenediyl-containing attachment group. Substrates having nitrile groups include, but are not limited to, coatings of polyacrylonitrile on a support material such as glass or a polymeric material. Suitable polymeric support material includes polyesters and polyimides, for example. Other suitable substrates having nitrile groups include acrylonitrile polymers or copolymers and 2-cyanoacrylate polymers or copolymers.

An azido group can also react with a strained olefinic group to form a triazolediyl-containing attachment group. Suitable substrates have a strained olefinic group include coatings that have pendant norbornenyl functional groups. Suitable support materials include, but are not limited to, glass and polymeric materials such as polyesters and polyimides.

An aziridinyl group can react with a mercapto group to form a aminoalkylthioether-containing attachment group. Examples of substrate materials having a mercapto group include, but are not limited to, mercapto substituted esters of polymethacrylates or polyacrylates and glass treated with a mercaptoalkylsilane.

Additionally, an aziridinyl group can react with a carboxy group to form a β-aminoalkyloxycarbonyl-containing attachment group. Suitable substrates having a carboxy include, but are not limited to, a coating of a acrylic acid polymer or copolymer, or a coating of a methacrylic acid polymer or copolymer on a glass or polymeric support. Copolymers include, but are not limited to, copolymers that contain polyethylene and polyacrylic acid or polymethacrylic acid. Suitable polymeric support materials include polyesters, polyimides, and the like.

A haloalkyl group can react, for example, with a substrate having a tertiary amino group to form a quaternary ammonium-containing attachment group. Suitable substrates having a tertiary amino group include, but are not limited to, polydimethylaminostyrene or polydimethylaminoethylmethacrylate.

Likewise, a tertiary amino group can react, for example, with a substrate having a haloalkyl group to form a quaternary ammonium-containing attachment group. Suitable substrates having a haloalkyl group include, for example, coatings of a haloalkylsilane on a support material. Support materials can include, but are not limited to, glass and polymeric materials such as polyesters and polyimides.

A primary aromatic amino or a secondary aromatic amino group can react, for example, with a substrate having an isocyanate group to form a oxycarbonylimino-containing attachment group. Suitable substrates having isocyanate groups include, but are not limited to, a coating of a 2-isocyanatoethylmethacrylate polymer or copolymer on a glass or polymeric support. Suitable polymeric supports include polyesters, polyimides, and the like.

A primary aromatic amino or a secondary aromatic amino group can also react with a substrate containing a carboxy or halocarbonyl group to form a carbonylimino-containing attachment group. Suitable substrates include, but are not limited to, acrylic or methacrylic acid polymeric coatings on a support material. The support material can be, for example, glass or a polymeric material such as polyesters or polyimides. Other suitable substrates include copolymers of polyethylene and polymethacrylic acid or polyacrylic acid.

A disulfide or an alkyl disulfide group can react, for example, with a metal surface to form a metal sulfide-containing attachment group. Suitable metals include, but are not limited to gold, platinum, palladium, nickel, copper, and chromium. The substrate can also be an alloy such an indium tin oxide or a dielectric material.

A benzotriazolyl can react, for example, with a substrate having a metal or metal oxide surface. Suitable metals or metal oxides include, for example, silver, aluminum, copper, chromium, iron, cobalt, nickel, zinc, and the like. The metals or metal oxides can include alloys such as stainless steel, indium tin oxide, and the like.

A phosphono, phosphoroamido, or phosphato can react, for example, with a substrate having a metal or metal oxide surface. Suitable metals or metal oxides include, for example, silver, aluminum, copper, chromium, iron, cobalt, nickel, zinc, and the like. The metals or metal oxides can include alloys such as stainless steel, indium tin oxide, and the like.

An ethylenically unsaturated group can react, for example, with a substrate having an alkyl group substituted with a mercapto group. The reaction forms a heteroalkylene-containing attachment group. Suitable substrates include, for example, mercapto-substituted alkyl esters of polyacrylates or polymethacrylates.

An ethylenically unsaturated group can also react with a substrate having a silicon surface, such as a silicon substrate formed using a chemical vapor deposition process. Such silicon surfaces can contain —SiH groups that can react with the ethylenically unsaturated group in the presence of a platinum catalyst to form an attachment group with silicon bonded to an alkylene group.

Additionally, an ethylenically unsaturated group can react with a substrate having a carbon-carbon double bond to form an alkylene-containing attachment group. Such substrates include, for example, polymers derived from butadiene.

A triazine moiety such as TCT can react with a nucleophile-containing materials including glass, diamond-like glass, metal and polymeric substrates with nucleophile functionality. Polymeric substrates can include, for example, ammonia grafted sintered polyethylene, aminated polyester blown melt fiber membrane, hydroxylated polypropylene, polyester, and polyethylene blown melt fiber membrane, and aminomethylated styrene divinylbenzene.

The compounds of Formula I can undergo a self-assembly process when contacted with a substrate. As used herein, the term “self-assembly” refers to process in which a material can spontaneously form a monolayer of tethering groups when contacted with a substrate.

Articles according to the invention typically include a substrate and a substrate-attached tethering group that includes a reaction product of a complementary substrate-functional group on a surface of the substrate with a triazine moiety, such as a compound of Formula I, where the substrate-attached functional group is a group capable of reacting with one of the X, Y or Z groups of Formula I to form an ionic bond, covalent bond, or combinations thereof.

More than one tethering group is typically attached to the substrate if there are more than one reactive group on the substrate. Further, the substrate can have excess reactive groups on the surface of the substrate that have not reacted with a tethering compound.

Groups on a substrate capable of reacting with a triazine group such as TCT or the X, Y or Z groups in compounds according to Formula I include, but are not limited to, hydroxy, mercapto, primary aromatic amino group, secondary aromatic amino group, secondary aliphatic amino group, azido, carboxy, carbonyloxycarbonyl, isocyanate, halocarbonyl, halocarbonyloxy, silanol, and nitrile.

The attachment of tethering compounds to the surface of a substrate (i.e., formation) can be detected using techniques such as, for example, contact angle measurements of a liquid on the substrate before and after attachment of a triazine tethering compound (e.g., the contact angle can change upon attachment of a tethering group to the surface of a substrate), ellipsometry (e.g., the thickness of the attached layer can be measured), time-of-flight mass spectroscopy (e.g., the surface concentration can change upon attachment of a tethering group to a substrate), and Fourier Transform Infrared Spectroscopy (e.g., the reflectance and absorbance can change upon attachment of a tethering group to a substrate).

In other embodiments of articles of the invention, a halogen-containing moiety in the tethering group has reacted with an amine-containing material resulting in the immobilization of an amine-containing material to the substrate. In some embodiments, the amine-containing materials are biomolecules such as, for example, amino acid, peptide, nucleoside or nucleotide, DNA or RNA oligonucleotide, DNA, RNA, PNA (peptide nucleic acid), protein, enzyme, organelle, immunoglobin, or fragments thereof. In other embodiments, the amine-containing material is a non-biological amine such as an amine-containing analyte. The presence of the immobilized amine can be determined, for example, using mass spectroscopy, contact angle measurement, infrared spectroscopy, and ellipsometry. Additionally, various immunoassays and optical microscopic techniques can be used if the amine-containing material is a biologically active material.

Other materials can be bound to the amine-containing material. For example, a complementary RNA or DNA fragment can hybridize with an immobilized RNA or DNA fragment. In another example, an antigen can bind to an immobilized antibody or an antibody can bind to an immobilized antigen. In a more specific example, a bacterium including gram positive bacteria and gram negative bacteria. In some embodiments, Staphylococcus aureus can bind to an immobilized biomolecule.

Another aspect of the invention provides methods for immobilizing a nucleophile-containing material to a substrate. The method involves preparing a substrate-attached tethering group by reacting a complementary functional group on the surface of the substrate with at least one of the reactive groups X, Y or Z in compounds of Formula I; and reacting at least one or more of the remaining reactive groups X, Y or Z of the substrate-attached tethering group with an nucleophile-containing material to form a triazine connector group between the substrate and the nucleophile-containing material. In one embodiment, the nucleophile-containing material is an amine-containing material and the method of immobilizing the amine-containing material is represented in Reaction Scheme A: 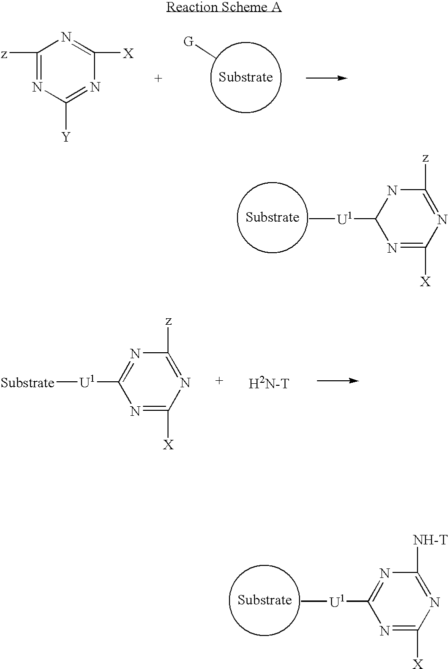
where U¹ is the attachment group formed by reacting X in compound of Formula I with a complementary functional group G on the surface of the substrate; T is the remainder of the amine-containing material, (i.e., the group T represents all of the amine-containing material exclusive of the amine group). The groups Y and Z are the same as previously defined for Formula I, and the foregoing Reaction Scheme will be understood to encompass reactions wherein X, Y and Z may be the same and are equally likely to react with a functional group G on the surface of the substrate. H₂N-T is any suitable amine-containing material. In some embodiments, H₂N-T is a biomolecule.

Variations of the foregoing Reaction Scheme A are also within the scope of the invention. In embodiments where monofunctional moieties are bonded to a triazine moiety, methods involve preparing a substrate-attached tethering group by reacting a complementary functional group on the surface of the substrate with at least one of the reactive groups X, Y or Z in compounds of Formula I, and reacting at least one or more of the remaining reactive groups X, Y or Z of the substrate-attached tethering group with one or more monofunctional moieties to form a tethering group that includes a triazine moiety bonded to a substrate with a monofunctional moiety also bonded to the triazine moiety. A nucleophile-containing material may be bonded to the triazine moiety to tether the nucleophile containing material to the substrate.

In embodiments having a difunctional moiety, the difunctional moiety is bonded to a first triazine moiety that is tethered to the surface of a substrate. The difunctional moiety may also be bonded to a second triazine moiety, and the second triazine moiety may be bonded to a nucleophile-containing material to tether the nucleophile-containing material to the substrate. In embodiments comprising multifunctional moieties, the multifunctional moiety may be bonded to a first triazine moiety that is tethered to the surface of a substrate and the multifunctional moiety also be bonded to a second, third, or other additional triazine moieties. In turn, the second or third or other triazine moiety may react with and bond to a nucleophile-containing material to tether the nucleophile-containing material to the substrate.

Accordingly, a method involves:

selecting a triazine compound of Formula I;

providing a substrate having a complementary functional group capable of reacting with X, Y or Z of the triazine compound of Formula I;

preparing a substrate-attached triazine moiety by reacting at least one of X, Y or Z of the triazine compound of Formula I with the complementary functional group on the substrate resulting in an ionic bond, covalent bond, or combinations thereof to form a triazine-containing connector group; and

reacting at least one of the unreacted groups X, Y or Z of the triazine-containing connector group with a nucleophile-containing material (e.g., an amine-containing material) to tether the nucleophile-containing material to the substrate.

In another aspect, a method involves:

selecting a triazine compound (e.g., a compound of Formula I);

providing a substrate having a complementary functional group capable of reacting with the triazine compound (e.g., with the X, Y or Z groups of Formula I);

preparing a substrate-attached triazine moiety by reacting the triazine moiety (e.g., at least one of X, Y or Z of Formula I) with the complementary functional group on the substrate resulting in an ionic bond, covalent bond, or combinations thereof; and

reacting the substrate-attached triazine moiety (e.g., at least one unreacted group X, Y or Z of Formula I) with a monofunctional, difunctional and/or multifunctional moiety to provide a triazine-containing connector group;

reacting the triazine-containing connector group (e.g., an unreacted group X, Y or Z of Formula I) the with a nucleophile-containing material (e.g., an amine-containing material) to tether the nucleophile-containing material to the substrate.

In another aspect, a method involves:

selecting a first triazine compound (e.g., a first compound of Formula I);

providing a substrate having a complementary functional group capable of reacting with the first triazine compound (e.g., X, Y or Z of Formula I);

preparing a substrate-attached triazine moiety by reacting the first triazine compound (e.g., one of X, Y or Z of Formula I) with a complementary functional group on the substrate resulting in an ionic bond, covalent bond, or combinations thereof;

reacting the substrate-attached triazine moiety (e.g., one of X, Y or Z of Formula I) with a difunctional and/or multifunctional moiety;

reacting the difunctional and/or multifunctional moiety with a second triazine compound (e.g., a second compound Formula I) to provide a triazine-containing tethering group;

reacting the triazine-containing tethering group (e.g., an unreacted group X, Y or Z of Formula I) with a nucleophile-containing material (e.g., an amine-containing material) to tether the nucleophile-containing material to the substrate.

The compounds of the invention can be used, for example, for immobilizing nucleophile-containing material such as an amine-containing material. In some embodiments, the amine-containing material is an amine-containing analyte. In other embodiments, the amine-containing materials are biomolecules such as, for example, amino acids, peptides, DNA, RNA, protein, enzymes, organelles, immunoglobins, or fragments thereof. Immobilized biological amine-containing materials can be useful in the medical diagnosis of a disease or of a genetic defect. The immobilized amine-containing materials can also be used for biological separations or for detection of the presence of various biomolecules. Additionally, the immobilized amine-containing materials can be used in bioreactors or as biocatalysts to prepare other materials. The substrate-attached tethering groups can be used to detect amine-containing analytes.

Biological amine-containing materials often can remain active after attachment to the substrate so that an immobilized antibody can bind with antigen or an immobilized antigen can bind to an antibody. An amine-containing material can bind to a bacterium. In a more specific example, the immobilized amine-containing material can bind to a Staphylococcus aureus bacterium (e.g., the immobilized amine-containing material can be a biomolecule that has a portion that can specifically bind to the bacterium).

Additional embodiments of the invention are described in the following non-limiting Examples.

### EXAMPLES

In the following Examples, substrates were utilized for subsequent reaction with triazine tethering compounds.

### Example 1

A TCT functionalized DLG-coated porous membrane was prepared. A 5 cm² high density polyethylene thermally induced phase separation (HDPE TIPS) membrane (3M Company, St. Paul, Minn.) with a pore size of about 0.09 μm and a thickness of about 0.9 mil (22.86 μm) was coated with diamond like glass (DLG) using a plasma process as described in Example 1 of U.S. Patent Application Publication No. 2003/0138619 (David et al.) to extend the DLG coating into the pores of the TIPS membrane. The DLG-coated TIPS membrane was placed in 50 ml of ethanol containing 2% by volume 3-amino propyl triethoxy silane (Sigma-Aldrich, St. Louis, Mo.), 1 ml water and few drops of 0.1N acetic acid. After 10 minutes in this solution the membrane was removed and washed with ethanol and dried.

The membrane was reacted with either TCT or a TCT oligomer for 1 hour at room temperature. A Sample A was created using 20 ml of a solution containing 0.2 g TCT and 36 g THF. The membrane was then washed five times with THF and dried and stored under N₂. A Sample B was created using 20 ml of a TCT oligomer at ˜7% solids in THF rolled in a vial for 1 hour with the membrane at room temperature. The TCT oligomer consisted of the reaction product of TCT and 4,7,10-trioxa-1,13-tridecanediamine (TOTDDA) in a 4/3 ratio. The 7 hour reaction at 4° C., with K₂CO₃ (29% in water), was expected to produce an oligomer with an average Mw of about 1180. The membrane was then washed five times with THF and dried and stored under N₂. A Sample C was created with 20 ml of a TCT oligomer at ˜9% solids in THF rolled overnight in a vial at room temperature with the membrane. This TCT oligomer was based on TCT and TOTDDA reacted with K₂CO₃ for 4 hours/4° C. in a 2/1 TCT/TOTDDA mole ratio in THF.

### Example 2

Samples were prepared as TCT functionalized NH₃ grafted POREX® polyethylene beads, as follows: Five (5) washed POREX® polyethylene beads (Porex Corporation—Fairburn, Ga.) were reacted with either TCT or a TCT oligomer for 1 hour at room temperature. Sample A was prepared with 1 ml of a solution containing 0.2 g TCT and 36 g THF. The beads were then washed five times with THF and dried and stored under N₂. Sample B was made with 1 ml of a TCT oligomer at ˜7% solids in THF rolled in a vial for 1 hour with 5 frits at room temperature. The TCT oligomer was the reaction product of TCT and 4,7,10-trioxa-1,13-tridecanediamine (TOTDDA) in a 4/3 TCT/TOTDDA mole ratio. The 7 hour reaction at 4° C., with K₂CO₃ (29% in water), was expected to produce an oligomer with an average Mw of about 1180. The beads were then washed five times with THF and dried and stored under N₂. Sample C was prepared with 2 ml of a TCT oligomer at ˜9% solids in THF rolled overnight in a vial at room temperature with 5 frits. This TCT oligomer was based on TCT and 4,7,10-trioxa-1,13-tridecanediamine (TOTDDA) reacted with K₂CO₃ for 4 hours/4° C. in a 2/1 TCT/TOTDDA mole ratio in THF.

Bis(hexamethylene)triamine was then added in a 1/3 mole ratio to the TCT/TOTDDA from the above sample C and reacted with K₂CO₃ for 2 hours at 23° C. to produce a TCT oligomer with an average Mw of about 1219. The beads were then washed five times with THF and dried and stored under N₂.

### Example 3

TCT functionalized membranes were prepared from aminated polyester blown melt fibers. Polyester non-woven membranes (3M Company, St. Paul, Minn.) were aminated with 3,3′-Iminobispropylamine (BASF, Mount Olive, N.J.). Each membrane was treated with TCT and Diisopropylethylamine (DIPEA) at fourteen times the amine level of membrane for 2 hours at room temperature. Membranes were then washed five times with THF and dried and stored under N₂.

### Example 4

TCT functionalized membranes were prepared from hydroxylated blown melt fibers. Polyester, polyethylene, and polypropylene non-wovens (3M Company, St. Paul Minn.) were oxidized in an aqueous solution of potassium peroxydisulfate (KPS) to yield the hydroxylated support. Hydroxylated polyester membranes were also prepared by base hydrolysis of the bulk membrane. Each membrane was treated with TCT and either NaOH (23° C./45 minutes in 20/80 water/acetone) or DIPEA (50° C./30 minutes in acetone). All membranes were then washed five times in acetone and air dried.

### Example 5

A section of EMPORE™ particle-loaded polytetrafluoroethylene (PTFE) membrane (3M Company, St. Paul, Minn.) containing aminomethylated styrene divinylbenzene (SDB) beads was treated with TCT or TCT oligomers. SBD Beads were aminomethylated in a two step procedure. Electrophilic aromatic substitution with N-hydroxymethyl phthalimide (Tscherniak-Einhorn reaction) was followed by treatment of the modified beads with alcoholic hydrazine hydrate. The procedure follows: the SDB beads (50 g) were suspended in 500 mL of a 1/1 (v/v) methylene chloride and trifluoroacetic acid mixture. Trifluoromethanesulfonic acid (4.65 mL) was added and the entire mixture was gently agitated at room temperature for 14 hours. The suspended beads were isolated by centrifugation and were washed with 100 ml each of 1/1 (v/v) methylene chloride and trifluoroacetic acid mixture, methylene chloride, ethanol, and methanol before being dried in vacuo. The reaction was checked by FT-IR spectroscopy. The resulting product was refluxed in 100 mL of 5% hydrazine hydrate in ethanol for 14 hours. The beads were again isolated by centrifugation before being washed with 1 L each of water, ethanol, and methanol. The beads were dried at 50° C. in vacuo to constant weight. The extent of aminomethylation was determined in triplicate by the ninhydrin assay. Sample D treated the film with TCT at three times the level of amine in the film, in cold THF and K₂CO₃ for 3 hours at 3° C. Sample E used a TCT oligomer consisting of TCT and TOTDDA 2/1 TCT/TOTDDA mole ratio for 2.5 hours at 2° C. then combining this with Bis(hexamethylene)triamine in 1/3 mole ratio to the above TCT/TOTDDA product at 2° C. and reacting overnight at 23° C. React with membrane for 3.5 hours at 23° C., washed five times with THF, dry and store under N₂. Sample F used ten times the TCT concentration to amine in the film for 2 hours at 23° C. with a five times THF rinse N₂ dry and store.

### Example 6

Conjugation of a 3′-NH₂ terminated DNA oligonucleotide capture probe can be performed directly on TCT derivatized materials. No additional activation steps are required prior to coupling of the amine terminated DNA oligonucleotide. Conjugation reactions were performed on 6 mm disks of the EMPORE membrane prepared in Example 5. For POREX solid supports, 1 frit was used per conjugation reaction. The membrane was transferred to a DNA conjugation solution containing 20-2000 pmol of a 3′-NH₂ terminated DNA oligonucleotide in 0.1 M Na₂HPO₄ (pH 8.5). The membranes were conjugated overnight at 4° C., removed from the conjugation solution, and rinsed with the following series of washes: H2O, 0.1M NaCl, H₂O, 0.1M NaOH, H₂O. The washed membranes (or frits) were stored at 4° C. until ready for use. The membranes were subsequently subjected to a prehybridization procedure using ethanol amine and/or bovine serum albumin (BSA). The purpose of the blocking solution is to minimize the occurrence of non-specific DNA binding.

### Example 7

A duplex sequencing reaction was performed utilizing two sequencing primers with a specific tag attached to each of the primers. A membrane prepared according to Example 6 was conjugated with the oligonucleotide complement to one of the sequencing primer tags as described in Example 6. The duplex sequencing reaction was passed through the membrane with the selective capture of sequencing ladders generated by only one of the sequencing primers. Subsequently, the sequencing ladders were released and sequenced on an ABI 377 or 310 sequencing instrument (Applera, Foster City, Calif.).

### Example 8

Six (6) μl of a duplex sequencing reaction in 14 μl hybridization buffer was loaded onto each membrane disk prepared as in Example 7 and incubated for 10 minutes at 42° C. After hybridization, the samples were centrifuged to remove excess sequencing reaction and washed twice with 75% isopropanol in H₂O and twice with 100% isopropanol. Thirty (30) μl of concentrated ammonium hydroxide was used to elute the hybridized sequencing ladder. The eluent was dried under vacuum and then resuspended in appropriate loading buffer depending on the instrument used for sequencing. Good quality sequencing data was obtained on an ABI 377 sequencing instrument.

### Example 9

A DLG coated TIPS membrane was prepared according to Example 1 to provide a substrate. The DLG/TIPS substrate was immersed for 10 minutes in a solution containing 45 ml ethanol, 2 ml water, 3 ml of 3-aminopropyltrimethoxy silane (Sigma-Aldrich, St. Louis, Mo.) and a few drops of acetic acid. After the 10 minute immersion, the membrane was washed three times with 100 ml aliquots of ethanol and then dried in an oven at 45° C. for one hour. A portion of the dried membrane was dipped in a 1% ninhydrin solution and oven dried at 45° C. to confirm the presence of primary amines on the surface of the support by the presence of a purple coloration.

A multifunctional moiety was made using a “PeOx” (poly 2-ethyl-2-oxazoline) polymer having a molecular weight of 5000 (Polymer Chemistry Innovations, Tucson, Ariz.). The PeOx (mol. wt. 5000) was dissolved at 25% solids in water in a 3 neck flask. A 38% solids solution of hydrochloric acid was added, such that the moles of HCl equal 22% of the moles of amide in the polymer. Flask was heated for 4.5 hours at 100 C. A condenser was used to trap the vapor into a separate flask, over the last 1.5 hours of the reaction. Remaining reaction content was poured into 3000 Mw cutoff dialysis membrane (Spectrum Labs, Rancho Dominquez, Calif.), with end clamps and stirred for 72 hours in a large beaker filled with deionized water. NaOH was used to keep the pH in the range of 9-10 and the deionized water was changed several times. The 20% hydrolyzed PeOx polymer solution was removed from the dialysis membrane, rotovaped and then vacuum oven dried at 60° C. to produce the solid polymer. One gram of the resulting amine containing polymer was then dissolved in 10 g cold THF (tetrahydrofuran) and 0.43 g DIPEA (diisopropylethylamine) and dripped into a flask containing 2.09 g of TCT dissolved in 15 g cold THF. The resulting reactive oligomer was precipitated from solution using heptane, rinsed in toluene and then redissolved in THF 3 times.

The reaction product was then reacted with 3-aminopropyltrimethoxy silane (13.8% solids in tetrahydrofuran) to provide reactive ligands pendant on the surface of the TIPS/DLG substrate. The substrate was then washed with THF several times and dried in a glove box under nitrogen at room temperature. Protein was then immobilized on the substrate by placing the substrate for 3 hours in a glucose oxidase solution (10 mg glucose oxidase in 5 ml of phosphate buffer). The membrane was removed and washed several times with water. A glucose oxidase assay confirmed that the substrate did bind the enzyme and was active.

The reaction scheme for the foregoing was as follows:

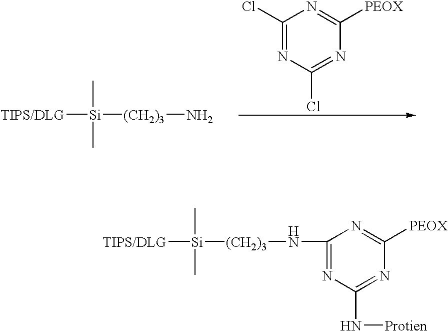

### Example 10

Glass slides were treated with DLG using the following conditions. Each glass slide was etched in oxygen plasma for 10 seconds and exposed to a mixture of tetramethylsilane and oxygen plasma for 20 seconds followed by oxygen plasma for another 10 seconds. The DLG coated glass slides were then placed in a 1% solution of 3-aminopropyltriethoxy silane in ethanol for 10 minutes. Thereafter, the glass slides were removed and washed with ethanol and dried under a nitrogen flow. Subsequently the glass slides were reacted with a solution of TCT in toluene (Sigma Aldrich, St. Louis, Mo.). The reaction time was varied.

The amine has a low contact angle of 20 degrees, which on reaction with TCT increases to 55 degrees. The sample was then reacted with 1 mM of lysine solution. On reaction with lysine (Sigma Aldrich), the contact angle decreases due to the reaction of the amino group of lysine to the TCT. This reaction was monitored with time. The contact angle decreased and stabilized within 10 mins of reaction. Contact angle data for the attachment of the TCT is provided in Table 1.

TABLE 1

| Time (min.) | Contact Angle |
|---|---|
| 5 | 26.3 |
| 20 | 25.3 |
| 30 | 20.5 |
| 60 | 13 |
| Overnight | 14 |

### Example 11

Gold was deposited by electron beam evaporation onto polyimide film. A 10 cm by 15 cm sample of polyimide film (available under the trade designation “KAPTON E” from E. I. Du Pont de Nemours & Co., Wilmington, Del.) was affixed to the plate of the planetary system in a Model Mark 50 high vacuum deposition system (available from CHA Industries, Fremont, Calif.) using metal stationery binder clips. The chamber was evacuated for approximately 2 hours, during which time the chamber pressure was reduced to approximately 6.7×10⁻⁴ Pa (5×10⁻⁶ mm Hg). Gold metal was deposited onto the polyimide film at a rate of approximately 1 Angstrom per second to a total thickness of approximately 2000 Angstroms. Deposition of gold was terminated and the system was allowed to cool for approximately 30 minutes before the chamber pressure was raised to atmospheric pressure and the samples were removed. The gold covered polyimide substrate was placed in aminothiophenol in toluene solution at a concentration of 1 mM. The sample was rinsed and dried after 10 min. Contact angle measurements revealed a contact angle of 68 degrees which increased to a contact angle of 75 degrees upon further reaction with TCT in a toluene solution. The sample was placed in a 1 mM lysine solution for different periods of time, rinsed and dried and additional contact angle measurements were taken. Contact angle measurements are set forth in Table 2.

TABLE 2

| Time (min.) | Contact Angle |
|---|---|
| 0 | 75 |
| 1 | 70.46 |
| 5 | 55.12 |
| 30 | 56.53 |
| 60 | 51.09 |

Similar samples were reacted with 1 mM Cysteine amino acid solution. The reaction of Cysteine to TCT would be through the thiol groups. This reaction was followed through the contact angle measurements set forth in Table 3.

TABLE 3

| Time (min.) | Contact Angle |
|---|---|
| 0 | 75 |
| 1 | 77.16 |
| 5 | 73.75 |
| 30 | 71.7 |
| 60 | 55.73 |

What is claimed is:

1. An article comprising:
a substrate having a first surface and a second surface;
a triazine tethering group affixed to the first surface of the substrate, the triazine tethering group comprising a reaction product of a functional group on the first surface of the substrate with a triazine tethering compound.

2. The article according to claim 1 wherein the first surface of the substrate comprises diamond-like glass.

3. The article according to claim 2 wherein the diamond-like glass is on a thermally induced phase separated membrane.

4. The article according to claim 3 wherein the thermally induced phase separated membrane comprises a material selected from the group consisting of high density polyethylene, polypropylene, polyvinylidenefluoride, polyethylene-vinyl alcohol copolymer and combinations of two or more of the foregoing.

5. The article according to claim 2 wherein the diamond-like glass is on glass.

6. The article according to claim 1 wherein the first surface of the substrate comprises polyethylene.

7. The article according to claim 6 wherein the polyethylene comprises aminomethylated polyethylene beads.

8. The article according to claim 1 wherein the first surface of the substrate comprises a blown melt fiber membrane comprising material selected from polyester, polypropylene, polyethylene and combinations of two or more of the foregoing.

9. The article according to claim 1 wherein the first surface of the substrate comprises aminomethylated styrene divinylbenzene beads incorporated in a polytetrafluoroethylene membrane.

10. The article according to claim 1, wherein the first surface of the substrate is a metal or a metal oxide selected from the group consisting of gold, silver, titanium, platinum, palladium, aluminum, copper, chromium, iron, cobalt, nickel, zinc, stainless steel, indium tin oxide, and combinations of two or more of the foregoing.

11. The article according to claim 10, wherein the substrate further comprises a support layer supporting the metal.

12. The article according to claim 11, wherein the support layer comprises a polymer.

13. The article according to claim 10, wherein the support layer comprises silicon.

14. The article according to claim 13 further comprising a tie layer between the silicon and the metal.

15. The article according to claim 1 wherein the triazine tethering compound comprises a structure according to Formula I 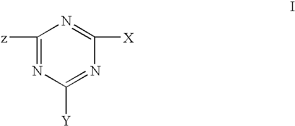
Wherein X, Y and Z may be the same or different and are radicals or organic or inorganic moieties capable of bonding with a nucleophile-containing material.

16. The article according to claim 15 wherein X, Y, and Z are chlorine.

17. The article according to claim 1 wherein the triazine tethering compound comprises a first triazine moiety and at least one of a monofunctional, difunctional or multifunctional moiety affixed to the first triazine moiety, the tethering group capable of bonding with a nucleophile-containing material.

18. The article according to claim 17 wherein the monofunctional moiety is selected from the group consisting of monofunctional organic alcohols, amines, mercaptans and combinations of two or more of the foregoing.

19. The article according to claim 17 wherein the difunctional moiety is bonded to the first triazine moiety and to a second triazine moiety, the difunctional moiety forming a linking group between the first and second triazine moieties.

20. The article according to claim 19 wherein the difunctional moiety is selected from the group consisting of 4,7,10-trioxa-1,13-tridecanediamine, 1,6-hexanediamine, methyl-oxirane, p-phenylenediamine, 2-aminoethanol, 4,4-thiobisbenzenethiol, dimethyl-1,6-hexanediamine and combinations of two or more of the foregoing.

21. The article according to claim 19 wherein the multifunctional moiety is bonded to the first triazine moiety and to one or more additional triazine moieties, the multifunctional moiety forming a linking group between the first triazine moiety and the one or more additional triazine moieties.

22. The article according to claim 21 wherein the multifunctional moiety is selected from the group consisting of hydrolyzed poly 2-ethyl-2-oxazoline, Bis(hexamethylene)triamine, polyethylenimine, hydroxy substituted esters of polymethacrylates, hydroxy substituted esters of polyacrylates, polyvinyl alcohol and combinations of two or more of the foregoing.

23. A method of immobilizing a nucleophile-containing material to a substrate, the method comprising:
Selecting a triazine tethering compound;
Providing a substrate having a complementary functional group capable of reacting with the triazine tethering compound;
Preparing a substrate-attached triazine tethering group by reacting the triazine tethering compound with the complementary functional group on the substrate resulting in an ionic bond, covalent bond, or combinations thereof; and
Reacting the substrate-attached triazine tethering group with a nucleophile-containing material to tether the nucleophile-containing material to the substrate.

24. The method according to claim 23 wherein the triazine tethering compound is a compound according to Formula I 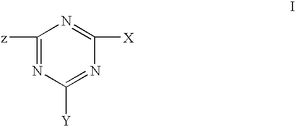
Wherein X, Y and Z may be the same or different and are radicals or organic or inorganic moieties capable of bonding with a nucleophile-containing material.

25. The method according to claim 24 wherein reacting the triazine tethering compound with the complementary functional group on the substrate comprises reacting at least one of X, Y or Z of a compound of Formula I with the complementary functional group to provide a substrate-attached triazine tethering group.

26. The method according to claim 25 wherein reacting the substrate-attached triazine tethering group with a nucleophile-containing material comprises reacting at least one unreacted group X, Y or Z of the substrate-attached tethering group with a nucleophile-containing material to tether the nucleophile-containing material to the substrate.

27. The method of claim 23, wherein the nucleophile-containing material is an amine-containing analyte, an amino acid, peptide, DNA, RNA, protein, enzyme, organelle, immunoglobulin, or fragment thereof.

28. The method of claim 23, wherein the nucleophile-containing material is an amine-containing material.

29. The method of claim 28, wherein the amine-containing material is an antigen and the antigen is further bound to an antibody.

30. The method of claim 28, wherein the amine-containing material is an immunoglobulin.

31. The method of claim 28, wherein the amine-containing material is further bound to a bacterium.

32. The method of claim 31, wherein the bacterium is selected from the group consisting of gram positive bacteria, gram negative bacteria, and combinations of the foregoing.

33. The method of claim 31, wherein the bacterium is Staphylococcus aureus

34. The method according to claim 23 further comprising reacting the substrate-attached triazine tethering group with a monofunctional, difunctional and/or multifunctional moiety, the monofunctional, difunctional or multifunctional moiety capable of bonding with the nucleophile-containing material to tether the nucleophile-containing material to the substrate.

35. The method according to claim 34 wherein the monofunctional moiety is selected from the group consisting of monofunctional organic alcohols, amines, mercaptans and combinations of two or more of the foregoing.

36. The method according to claim 34 wherein the difunctional moiety is selected from the group consisting of 4,7,10-trioxa-1,13-tridecane diamine, 1,6-hexanediamine, methyl-oxirane, p-phenylenediamine, 2-aminoethanol, 4,4-thiobisbenzenethiol, dimethyl-1,6-hexanediamine and combinations of two or more of the foregoing.

37. The method according to claim 34 wherein the multifunctional moiety is selected from the group consisting of hydrolyzed poly 2-ethyl-2-oxazoline, Bis(hexamethylene)triamine, polyethylenimine, hydroxy substituted esters of polymethacrylates, hydroxy substituted esters of polyacrylates, polyvinyl alcohol and combinations of two or more of the foregoing.
